(19) United States
(12) Patent Application Publication
(10) Pub. No.: US 2017/0170419 A1
(43) Pub. Date: Jun. 15, 2017

(54) ORGANIC FLUORESCENT LUMINESCENT MATERIAL AND ORGANIC ELECTROLUMINESCENT DEVICE

(71) Applicant: AU OPTRONICS CORPORATION, Hsin-chu (TW)

(72) Inventors: Hung-Chieh HU, Hsin-chu (TW); Chung-Chia CHEN, Hsin-chu (TW); Meng-Ting LEE, Hsin-chu (TW); Yu-Yi HO, Hsin-chu (TW); Shu-Wei LI, Hsin-chu (TW); Ken-Tsung WONG, Hsin-chu (TW); Kuan-Chung PAN, Hsin-chu (TW); Yi-Jiun SHIU, Hsin-chu (TW); Wei-Lung TSAI, Hsin-chu (TW); Chung-Chih WU, Hsin-chu (TW)

(21) Appl. No.: 15/374,197

(22) Filed: Dec. 9, 2016

(30) Foreign Application Priority Data
Dec. 11, 2015 (TW) 104141714

(51) Int. Cl. H01L 51/50; C09K 11/06; H01L 51/00; C07D 401/10

(52) U.S. Cl. CPC H01L 51/5012; C07D 401/10; C09K 11/06; H01L 51/0072; H01L 51/0067; H01L 2251/556; C09K 2211/1007; C09K 2211/1029; C09K 2211/1044

(57) ABSTRACT

An organic fluorescent luminescent material and an organic electro-luminescence device are provided. The organic fluorescent luminescent material may be represented by the formula, (Ar)-(Ph)-(X), wherein Ar is

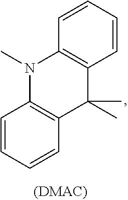 [SMILES: CN1c2ccccc2C(C)(C)c2ccccc21]

Ph include at least one benzene ring, X is a six-membered aromatic ring, and at least one nitrogen atom is located on the six-membered aromatic ring. The organic fluorescent luminescent material can be used in organic electro-luminescence devices.

### CROSS-REFERENCE TO RELATED APPLICATION

This non-provisional application claims priority to and benefit of, under 35 U.S.C. §119(a), Patent Application No. 104141714 filed in Taiwan R.O.C. on Dec. 11, 2015, the entire contents of which are hereby incorporated by reference. Some references, which may include patents, patent applications and various publications, are cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### TECHNICAL FIELD

The present disclosure relates to an organic fluorescent luminescent material and organic electroluminescent device, and more particularly to an organic fluorescent luminescent material and organic electroluminescent device based on thermal activated delayed fluorescence mechanism.

### BACKGROUND

With the development of active matrix organic light-emitting diode (AMOLED) technique, how to effectively improve the efficiency of a device has become an issue of high concern in the art, wherein the design of a luminescent material layer in the device determines the efficiency of the device. Generally, based on the results of spin-statistics, the ratio of singlet excitons and triplet excitons generated by recombination of electrons and holes is 1:3, so the maximum efficiency available by an OLED device with phosphorescence as emission mechanism can be 4 times that of a fluorescent OLED device. However, because the phosphorescent OLED device uses a complex containing a heavy metal such as Ir, Pt, or Os as light emitter, not only the manufacturing cost of the device itself is increased, but also the lifetime of these heavy metal light emitters cannot be effectively increased, so that the phosphorescent OLED device cannot be successfully used in various displays. On the other hand, although the fluorescent OLED device has stable performance, since the proportion of singlet excitons is low, the maximum external quantum efficiency of a conventional fluorescent OLED device is generally considered to be only 5%. In view of this, how to retransform triplet excitons into singlet excitons through other mechanisms to improve the overall fluorescence emission efficiency is still an issue to be addressed in the art.

### SUMMARY

An object of the present disclosure is to provide an organic fluorescent luminescent material and an organic electroluminescent device, by which triplet excitons are retransformed into singlet excitons to improve the overall fluorescence emission efficiency.

One embodiment of the present disclosure provides an organic fluorescent luminescent material represented by the formula (Ar)-(Ph)-(X), wherein Ar is

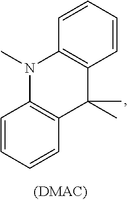 [SMILES: CN1c2ccccc2C(C)(C)c2ccccc21]

Ph comprises at least one benzene ring, X is a six-membered aromatic ring, and at least one nitrogen atom is located on the six-membered aromatic ring.

One embodiment of the present disclosure provides an organic fluorescent luminescent material represented by the formula (Ar)-(Ph)-(X), wherein Ar is

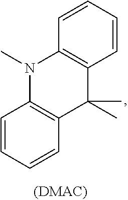 [SMILES: CN1c2ccccc2C(C)(C)c2ccccc21]

Ph comprises at least one benzene ring, X is a six-membered aromatic ring having positions 1, 2, 3, 4, 5 and 6, and at least one nitrogen atom is located on positions 1, 2, 3, 4 or 5, and the organic fluorescent luminescent material is represented by the formula as below:

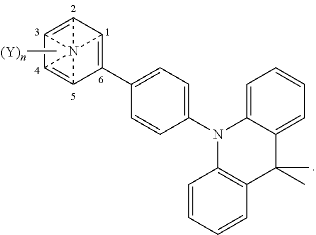

Another embodiment of the present disclosure provides an organic electroluminescent device, comprising an anode, a hole transport layer, an emissive layer, an electron transport layer, and a cathode. The hole transport layer is disposed on the anode. The emissive layer is disposed on the hole transport layer, wherein the emissive layer comprises the organic fluorescent luminescent material as described above. The electron transport layer is disposed on the emissive layer. The cathode is disposed on the electron transport layer.

The organic fluorescent luminescent material of the present disclosure can improve the overall fluorescence emission efficiency through thermal activated delayed fluorescence mechanism by retransforming triplet excitons into singlet excitons, such that the emission efficiency and lifetime of the organic electroluminescent device is effectively improved.

### BRIEF DESCRIPTION OF THE DRAWINGS

FIG. 1 shows fluorescence spectra of organic fluorescent luminescent materials of Examples 1-5 of the present disclosure in film state.

FIG. 2 shows transient spectra of organic fluorescent luminescent materials of Examples 1-5 of the present disclosure in film state at 300 k.

FIG. 3 shows a cross-sectional schematic view of an organic electroluminescent device of the present disclosure.

FIG. 4 shows a color coordinate diagram of organic electroluminescent devices of Experimental Examples 1-5 of the present disclosure.

### DETAILED DESCRIPTION

In order to allow a person of ordinary skill in the art to further understand the features and technical content of the present disclosure, reference can be made to the detailed description and accompanying drawings of the present disclosure.

The present disclosure provides an organic fluorescent luminescent material represented by the formula (Ar)-(Ph)-(X), wherein Ar is

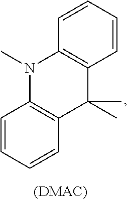 [SMILES: CN1c2ccccc2C(C)(C)c2ccccc21]

Ph comprises at least one benzene ring, X is a six-membered aromatic ring, and at least one nitrogen atom is located on the six-membered aromatic ring.

Preferably, the organic fluorescent luminescent material may be represented by the formula (I):

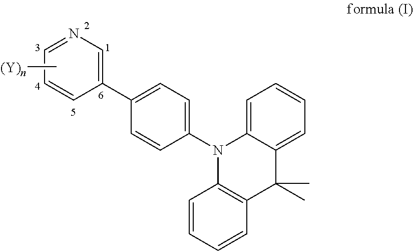

wherein nitrogen atom is located on position 2 so that X is pyridine, and Y is halogen, a trifluoromethyl group (—CF₃), or a cyanide group (—CN), and n is an integer from 1 to 4.

More preferably, in the formula (I) Y is the cyanide group (—CN) and n is 1, and the organic fluorescent luminescent material may be represented by the formula (II):

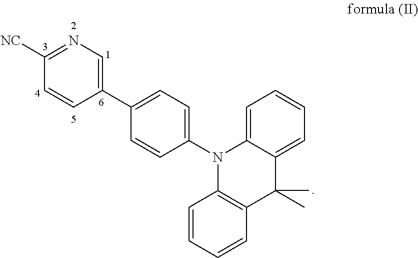 [SMILES: CC1(C)c2ccccc2N(c2ccc(-c3ccc(C#N)nc3)cc2)c2ccccc21]

wherein nitrogen atom is located on position 2 so that X is a pyridine, and the cyanide group is located on position 3.

Preferably, the organic fluorescent luminescent material may be represented by the formula (III):

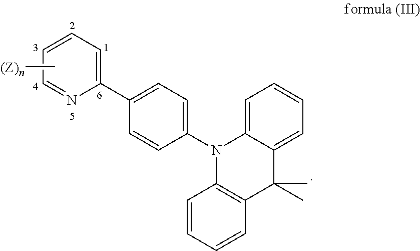

wherein nitrogen atom is located on position 5 so that X is pyridine, and Z is halogen, a trifluoromethyl group (—CF₃), or a cyanide group (—CN), and n is an integer from 1 to 4.

More preferably, in the formula (III) Z is the cyanide group (—CN) and n is 1, and the organic fluorescent luminescent material may be represented by the formula (IV):

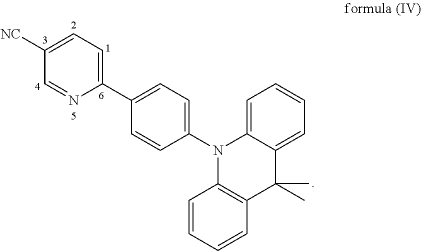 [SMILES: CC1(C)c2ccccc2N(c2ccc(-c3ccc(C#N)cn3)cc2)c2ccccc21]

wherein nitrogen atom is located on position 5 so that X is pyridine, and the cyanide group is located on position 3.

More preferably, in the formula (III) Z is the cyanide group (—CN) and n is 2, and the organic fluorescent luminescent material may be represented by the formula (V):

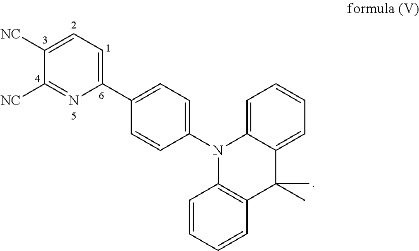 [SMILES: CC1(C)c2ccccc2N(c2ccc(-c3ccc(C#N)c(C#N)n3)cc2)c2ccccc21]

wherein nitrogen atom is located on position 5 so that X is pyridine, and the cyanide groups are located on positions 3 and 4.

Preferably, the organic fluorescent luminescent material may be represented by the formula (VI):

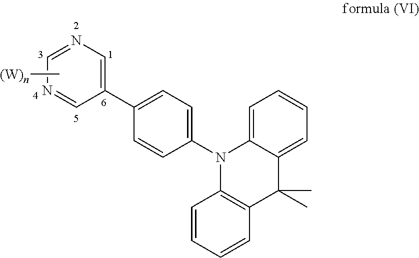

wherein nitrogen atoms are located on positions 2 and 4 so that X is diazine, and W is halogen, a trifluoromethyl group (—CF₃), or a cyanide group (—CN), and n is an integer from 1 to 3.

More preferably, in the formula (VI) W is the cyanide group (—CN) and n is 1, and the organic fluorescent luminescent material may be represented by the formula (VII):

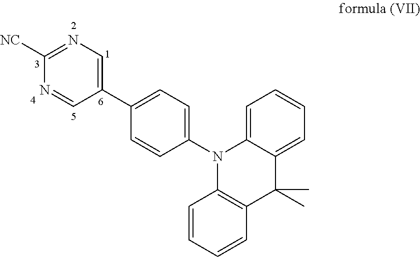 [SMILES: CC1(C)c2ccccc2N(c2ccc(-c3cnc(C#N)nc3)cc2)c2ccccc21]

wherein nitrogen atoms are located on positions 2 and 4 so that X is diazine, and the cyanide group is located on position 3.

Preferably, the organic fluorescent luminescent material may be represented by the formula (VIII):

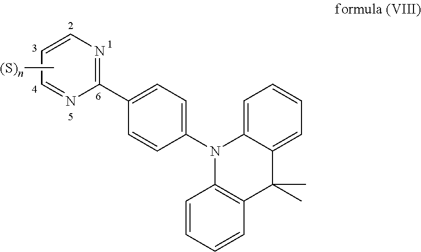

wherein nitrogen atom are located on position 1 and 5 so that X is diazine, and S is halogen, a trifluoromethyl group (—CF₃), or a cyanide group (—CN), and n is an integer from 1 to 3.

More preferably, in the formula (VIII) S is the cyanide group (—CN) and n is 1, and the organic fluorescent luminescent material may be represented by the formula (IX):

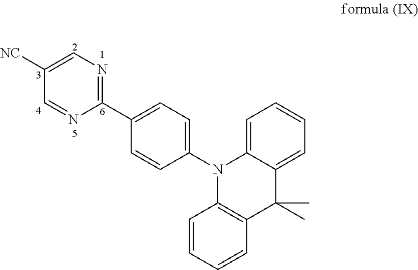 [SMILES: CC1(C)c2ccccc2N(c2ccc(-c3ncc(C#N)cn3)cc2)c2ccccc21]

wherein nitrogen atoms are located on position 1 and 5 and the cyanide group is located on position 3.

Experimental testing apparatus and conditions:

¹H nuclear magnetic resonance (abbreviated ¹H NMR) is performed by Varian 400 unity plus (400 MHz) apparatus. Sample solvents are chloroform-d(CDCl₃) and Dichloromethane-d2(CD₂Cl₂), the chemical shift is in ppm, chloroform(CHCl₃) as internal standard in ¹H NMR has a chemical shift of 7.26, and dichloromethane(CH₂Cl₂) as internal standard in ¹H NMR has a chemical shift of 5.32. The splitting pattern is defined as follows: s, singlet; d, doublet; t, triplet; q, quartet; qn, quintet; sex, sextet; m, multiplet; dd, doublet of doublets. The coupling constant is represented by J, in unit Hz. Spectral data is recorded in order: chemical shift, splitting pattern, coupling constant, and hydrogen number.

¹³C nuclear magnetic resonance (abbreviated ¹³C NMR) is performed by the same type apparatus Varian 400 MHz, in which the chemical shift is in ppm, the shift of the internal standard CDCl₃ δ=77.0, and the shift of the internal standard CD₂Cl₂ δ=54.0.

Infrared spectroscopy (abbreviated IR) is performed by Varian FT-IR type apparatus, in which a liquid sample is applied onto a sodium chloride (NaCl) tablet in neat form, a solid sample is prepared into a potassium bromide (KBr) sheet to be tested, and the results are expressed in wavenumber (cm⁻¹).

High-resolution mass spectroscopy (abbreviated HRMS) is performed by JEOL SX-102A type high-performance mass spectrometer and Shimadzu LCMS-IT-TOF mass spectrometer. Data is recorded in order: Fast atom bombardment (FAB⁺) and mass to charge ratio (m/z).

Elemental analysis is performed by Heraeus VarioEL type apparatus.

Melting point (abbreviated mp) is determined by Fargo MP-1D apparatus or read from differential scanning calorimetry (abbreviated DSC) spectra.

Thermal analysis is performed by TA Instrument 5100.

Ultraviolet-visible absorption spectrometry: absorption spectra are measured by using JASCO V-670 spectrophotometer as a spectrophotometer, in which using quartz cells with an optical path length of 1 cm, two quartz cells containing the same solvent are simultaneously placed in a reference optical path to correct the base line of absorption spectra, and then a sample solution is substituted for the solvent in one of the two quartz cells to measure the absorption spectrum of the sample solution. Generally, concentrations of samples used is five values from 5×10⁻⁵ M to 5×10⁻⁷ M, i.e. 5×10⁻⁵, 1×10⁻⁵, 5×10⁻⁶, 1×10⁻⁶, and 5×10⁻⁷ M, respectively.

Fluorescence spectral measurement: fluorescence spectra are measured by a fluorescence spectral scanner F-4500 (HITACHI, Japan), equipped with a monochromator in both excitation and emission paths.

Thin-layer chromatography (abbreviated TLC) is performed by using Merck 25 TLC (1.05554.0001, silica gel 60 F₂₅₄) as silica gel sheet for development with a UV lamp for inspection.

Column chromatography is performed by using Merck (0.063-0.200 mm) or (40-63 μm) silica gel. Eluent is formulated in a volume ratio in case of a bi-solvent system, and recorded in vol % of solvent A and vol % of solvent B. All solvents and reagents are reagent chemicals.

Diethyl ether and oxolane(THF) are dried over metallic sodium, with benzophenone anion radical as indicator, and distilled for use. N,N-dimethyl formamide (DMF) is dried over CaH₂, and toluene and CH₂Cl₂ are dried over P₂O₅. All reactions are stirred using a magnetic stirring bar. Reactions by heating at reflux all are operated in an argon or nitrogen system.

The present disclosure is further explained below through specific embodiments, but the present disclosure is not limited thereto.

### (1) synthesis of precursors (4-(9,9-dimethylacridin-10(9H)-yl)phenyl)boronic acid (DMAC—P—B(OH)₂) and 5-bromopyrimidine-2-carbonitrile

DMAC—P—B(OH)2 and 2-CN-5-BrPy were prepared with reference to J. Am. Chem. Soc., 2014, 136 (52), pp 18070-18081 and J. Med. Chem., 2013, 56 (2), pp 568-583 respectively, and represented by the following formulae respectively.

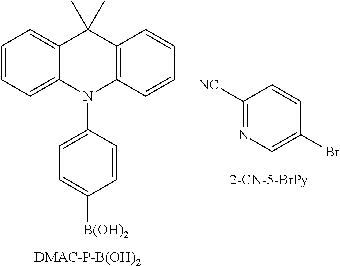

### (2) synthesis of precursor 6-chloropyridine-2,3-dicarboxamide

Firstly, a 100 mL single-necked flask was added with dimethyl 6-chloropyridine-2,3-dicarboxylate (8.41 g, 36.62 mmol) and NH₄Cl (0.33 g, 6.17 mmol) and equipped with a magnetic stirring bar. NH₄OH (53 mL) was slowly added and stirred at room temperature (RT) for 24 hours. The powder was precipitated and filtered to give a light pink solid foam. Then, a 250 mL two-necked flask was added with 6-chloropyridine-2,3-dicarboxamide (3.71 g, 18.58 mmol), equipped with a magnetic stirring bar, an addition funnel, and a condensing tube, and charged with argon after evacuation. Dry dimethylformamide (DMF) (60 mL) was firstly injected with a syringe, and then thioyl chloride (3 mL, 40.87 mmol) was injected with a syringe into the addition funnel, slowly added to the reaction flask at 0° C., stirred at 0° C. for two hours, allowed to gradually cool down to RT, and further stirred for 12 hours. After the reaction was deemed complete by TLC, it was slowly poured to icy water to terminate the reaction. It was extracted with ethyl acetate followed by saturated saline once, and then dried over anhydrous magnesium sulfate. After filtration, the filtrate was concentrated under reduced pressure, and the solid was purified by column chromatography (column filler SiO₂, mixture Ethyl Acetate/Hexane=3/4), giving a white solid (3.7 g, 4.42 mmol, 80%). Melting point (mp) 196° C.; ¹H NMR (400 MHz, Deuterated chloroform (CDCl₃)) chemical shift (δ) (ppm) 8.07 (splitting pattern (d), coupling constant (J)=8.4 Hz, hydrogen number (H)=1), 7.72 (d, J=8.4 Hz, 1H). ¹³C NMR (100 MHz, CDCl₃) δ (ppm) 142.42, 128.50. HRMS (FAB⁺, m/z) calcd for C₇H₂ClN₃, 162.9937; found 162.9944. The preparation process is shown below.

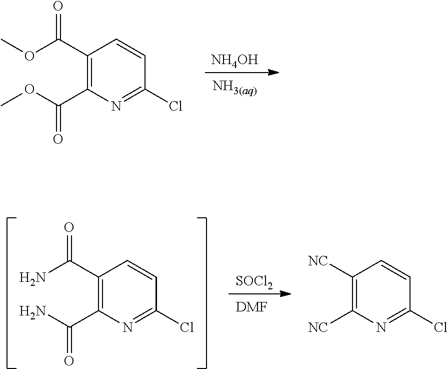

### (3) synthesis of 2-chloropyrimidine-5-carbonitrile

A 500 mL two-necked flask was added with 5-bromopyrimidin-2-amine (5.18 g, 30 mmol), Zn(CN)₂ (2.11 g, 18 mmol), Pd(PPh₃)₄ (3.46 g, 3 mmol), Zinc (0.045 g, 1.5 mmol), and Zn(OAc)₂ (0.33 g, 1.5 mmol), equipped with a magnetic stirring bar, an addition funnel, and a condensing tube, and charged with argon after evacuation. Dry DMF (300 mL) was injected with a syringe, heated at 120° C. for 24 hours, allowed to cool down to RT, and poured into 1000 mL water. The white powder was precipitated, filtered, and washed with methanol, giving a white powder formed 2-aminopyrimidine-5-carbonitrile. Then, a 100 mL two-necked flask was added with 2-aminopyrimidine-5-carbonitrile (3.6 g, 30 mmol), tert-butyl nitrite (BuNO) (5.35 mL, 43 mmol), equipped with a magnetic stirring bar, an addition funnel, and a condensing tube, and charged with argon after evacuation. Acetonitrile (60 mL) was firstly injected with a syringe, stirred at RT for 1 hour, and then a solid CuCl₂ was added, heated at 60° C. for 24 hours. After cool down to RT, 20 mL diethyl ether was added and filtered. After the filtrate was subjected to rotary concentration to remove excess solvent, the solid was purified by column chromatography (SiO₂, Dichloromethane/Hexane=1/1), giving a white solid (3 g, 22 mmol, 72%). ¹H NMR (400 MHz, CDCl₃) δ ppm 8.90 (s, 2H). The preparation process is shown below.

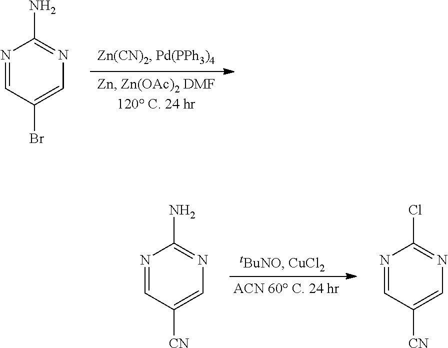 [SMILES: Nc1ncc(Br)cn1 ; N#Cc1cnc(N)nc1 ; N#Cc1cnc(Cl)nc1]

### Example 1: Synthesis of Formula (II)

A 25 mL two-necked flask was added with 5-bromopicolinonitrile (0.31 g, 1.69 mmol), DMAC—P—B(OH)₂ (0.62 g, 1.86 mmol), and Pd(PPh₃)₄ (98 mg, 0.28 mmol), equipped with a magnetic stirring bar and a condensing tube, and charged with argon after evacuation. Then, P^{t}Bu₃ (3.5 mL, 0.05 M), toluene (5 mL), and K₂CO_{3(aq)} (3.5 mL, 2.5 M) were injected with a syringe, stirred, and heated to reflux, and cooled to RT after the reaction was stirred for 48 hours. The salts were filtered out with diatomite, and extraction was performed with water and ethyl acetate followed by dichlormethane. Finally, extraction was performed with saturated saline. The organic layer was dried over anhydrous magnesium sulfate, and filtered. The filtrate was concentrated under reduced pressure, and the solid was purified by column chromatography (SiO₂, CH₂Cl₂/Hexane=2/1), giving a yellow solid (0.51 g, 1.31 mmol, 77%). ¹H NMR (400 MHz, CDCl₃) δ (ppm) 9.06 (d, J=2.2 Hz, 1H), 8.12 (dd, J=8.1, 2.3 Hz, 1H), 7.85 (dd, J=12.4, 8.2 Hz, 3H), 7.57-7.43 (m, 4H), 7.04-6.87 (m, 4H), 6.38-6.24 (m, 2H), 1.71 (s, 6H). ¹³C NMR (100 MHz, CDCl₃) δ (ppm) 149.64, 142.72, 140.60, 138.96, 135.67, 134.99, 132.67, 132.51, 130.29, 129.81, 128.50, 126.39, 125.40, 120.93, 117.22, 113.97, 36.02, 31.23. HRMS (FAB⁺, m/z) calcd for C₂₇H₂₁N₃, 387.1735; found 387.1744. The preparation process is shown below.

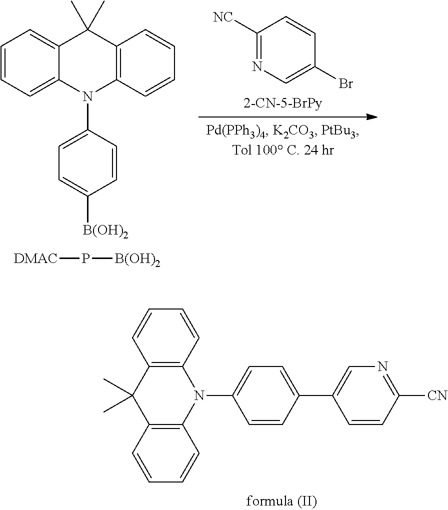

### Example 2: Synthesis of Formula (IV)

A 50 mL two-necked flask was added with 6-bromonicotinonitrile (0.55 g, 3 mmol), DMAC—P—B(OH)₂ (1.1 g, 3.3 mmol), and Pd(PPh₃)₄ (173 mg, 0.15 mmol), equipped with a magnetic stirring bar and a condensing tube, and charged with argon after evacuation. Then, P^{t}Bu₃ (6 mL, 0.05 M), toluene (9 mL), and K₂CO_{3(aq)} (6 mL, 2.5 M) were injected with a syringe, stirred, and heated to reflux, and cooled to RT after the reaction was stirred for 48 hours. The salts were filtered out with diatomite, and extraction was performed with water and ethyl acetate followed by dichlormethane. Finally, extraction was performed with saturated saline. The organic layer was dried over anhydrous magnesium sulfate, and filtered. The filtrate was concentrated under reduced pressure, and the solid was purified by column chromatography (SiO₂, CH₂Cl₂/Hexane=2/1), giving a yellow solid (1.15 g, 2.96 mmol, 98%). ¹H NMR (400 MHz, CDCl₃) δ (ppm) 9.00 (dd, J=2.1, 0.8 Hz, 1H), 8.39-8.25 (m, 2H), 8.08 (dd, J=8.3, 2.2 Hz, 1H), 7.96 (dd, J=8.3, 0.8 Hz, 1H), 7.59-7.42 (m, 4H), 6.93-6.98 (m, 4H), 6.42-6.25 (m, 2H), 1.70 (s, 6H). ¹³C NMR (CD2Cl2, 100 MHz) δ (ppm) 159.63, 152.57, 143.69, 140.58, 140.06, 137.11, 132.00, 130.26, 129.90, 126.38, 125.34, 120.86, 120.12, 116.83, 114.06, 108.26, 36.01, 31.23. HRMS (FAB⁺, m/z) calcd for C₂₇H₂₁N₃, 387.1735; found 387.1740. The preparation process is shown below.

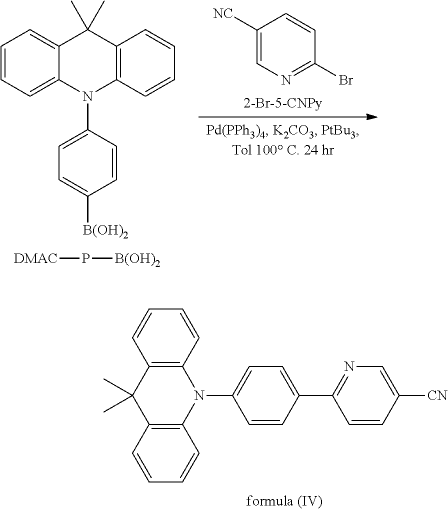

### Example 3: Synthesis of Formula (V)

A 100 mL two-necked flask was added with 6-chloropyridine-2,3-dicarboxamide (0.981 g, 6 mmol), DMAC—P—B(OH)₂ (2.17 g, 6.6 mmol), and Pd(PPh₃)₄ (346 mg, 0.3 mmol), equipped with a magnetic stirring bar and a condensing tube, and charged with argon after evacuation. Then, P^{t}Bu₃ (12 mL, 0.05 M), toluene (18 mL), and K₂CO_{3(aq)} (12 mL, 2.5 M) were injected with a syringe, stirred, and heated to reflux, and cooled to RT after the reaction was stirred for 48 hours. The salts were filtered out with diatomite, and extraction was performed with water and ethyl acetate followed by dichlormethane. Finally, extraction was performed with saturated saline. The organic layer was dried over anhydrous magnesium sulfate, and filtered. The filtrate was concentrated under reduced pressure, and the solid was purified by column chromatography (SiO₂, CH₂Cl₂/Hexane=2/1), giving a yellow solid (1.95 g, 4.72 mmol, 78%). ¹H NMR (400 MHz, CDCl₃) δ (ppm) 8.34 (d, J=8.5 Hz, 2H), 8.21 (d, J=8.4 Hz, 1H), 8.16 (d, J=8.4 Hz, 1H), 7.55 (d, J=8.5 Hz, 2H), 7.49 (d, J=9.0 Hz, 2H), 6.98 (dd, J=7.4, 1.9 Hz, 4H), 6.32 (d, J=7.6 Hz, 2H), 1.70 (s, 6H). ¹³C NMR (100 MHz, CDCl₃) δ (ppm) 160.50, 145.05, 141.15, 140.42, 136.50, 134.83, 131.99, 130.67, 130.17, 126.40, 125.41, 122.55, 121.15, 114.24, 113.87, 112.27, 36.07, 31.14. HRMS (FAB⁺, m/z) calcd for C₂₈H₂₀N₄, 412.1688; found 412.1688. The preparation process is shown below.

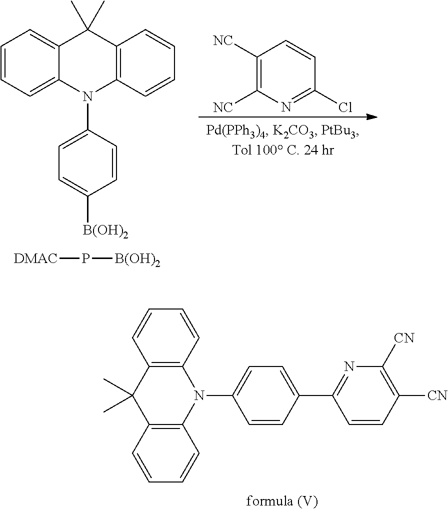

### Example 4: Synthesis of Formula (VII)

A 50 mL two-necked flask was added with 2-CN-5-BrPy (0.22 g, 1.2 mmol), DMAC—P—B(OH)₂ (0.33 g, 1 mmol), Pd(PPh₃)₄ (120 mg, 0.1 mmol), [1,1′-biphenyl]-2-yldicyclohexylphosphine) (0.035 g, 0.1 mmol), equipped with a magnetic stirring bar and a condensing tube, and charged with argon after evacuation. Then, toluene (20 mL) and K₂CO_{3(aq)} (1.6 mL, 2 M) were injected with a syringe, stirred, and heated to reflux, and cooled to RT after the reaction was stirred for 24 hours. The salts were filtered out with diatomite, and extraction was performed with water and ethyl acetate followed by dichlormethane. Finally, extraction was performed with saturated saline. The organic layer was dried over anhydrous magnesium sulfate, and filtered. The filtrate was concentrated under reduced pressure, and the solid was purified by column chromatography (SiO₂, CH₂Cl₂/Hexane=2/1), giving a yellow solid (0.36 g, 0.93 mmol, 93%). ¹H NMR (400 MHz, CD₂Cl₂) δ (ppm) 9.16 (s, 2H), 7.91 (d, J=8.4 Hz, 2H), 7.55 (d, J=8.4 Hz, 2H), 7.47 (d, J=8.4 Hz, 2H), 6.94 (quin), J=3.2 Hz, 4H), 6.29 (d, J=8.0 Hz, 2H), 1.66 (s, 6H). ¹³C NMR (100 MHz, CD₂Cl₂) δ (ppm) 156.54, 144.24, 144.02, 141.19, 136.29, 133.23, 133.08, 131.05, 130.46, 126.92, 125.96, 121.53, 116.50, 114.61, 66.58, 36.55, 31.54, 31.16. HRMS (FAB⁺, m/z) calcd for C₂₆H₂₀N₄₄, 388.1688; found 388.1685. Anal. calcd (%) for C₂₆H₂₀N₄₄: C, 80.39; H, 5.19; N, 14.42; found: C, 80.40; H, 5.15; N, 14.31. The preparation process is shown below.

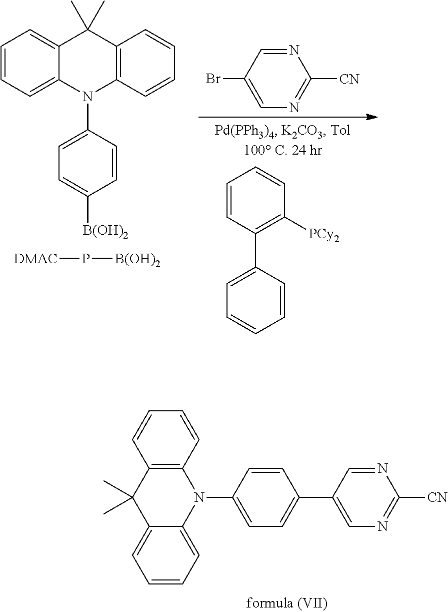

### Example 5: Synthesis of Formula (IX)

A 50 mL two-necked flask was added with 2-chloropyrimidine-5-carbonitrile (0.16 g, 1.2 mmol), DMAC—P—B(OH)₂ (0.33 g, 1 mmol), Pd(PPh₃)₄ (120 mg, 0.1 mmol), [1,1′-biphenyl]-5-yldicyclohexylphosphine) (0.035 g, 0.1 mmol), equipped with a magnetic stirring bar and a condensing tube, and charged with argon after evacuation. Then, toluene (20 mL) and K₂CO_{3(aq)} (1.6 mL, 2 M) were injected with a syringe, stirred, and heated to reflux, and cooled to RT after the reaction was stirred for 24 hours. The salts were filtered out with diatomite, and extraction was performed with water and ethyl acetate followed by dichlormethane. Finally, extraction was performed with saturated saline. The organic layer was dried over anhydrous magnesium sulfate, and filtered. The filtrate was concentrated under reduced pressure, and the solid was purified by column chromatography (SiO₂, CH₂Cl₂/Hexane=2/1), giving a yellow-orange solid (0.32 g, 0.82 mmol, 82%). ¹H NMR (400 MHz, CD₂Cl₂) δ (ppm) 9.09 (s, 2H), 8.75 (d, J=8.4 Hz, 2H), 7.51 (m, 4H), 6.93 (quin), J=3.2 Hz, 4H), 6.34 (d, J=8.0 Hz, 2H), 1.67 (s, 6H). ¹³C NMR (CD2Cl2, 100 MHz) δ (ppm) 166.04, 160.64, 146.07, 141.17, 136.10, 132.27, 131.99, 131.24, 126.93, 125.88, 121.54, 115.54, 114.90, 107.80, 36.60, 31.46. HRMS (FAB⁺, m/z) calcd for C₂₆H₂₀N₄₄, 388.1688; found 388.1689. Anal. calcd (%) for C₂₆H₂₀N₄₄: C, 80.39; H, 5.19; N, 14.42; found: C, 80.40; H, 5.21; N, 14.21. The preparation process is shown below.

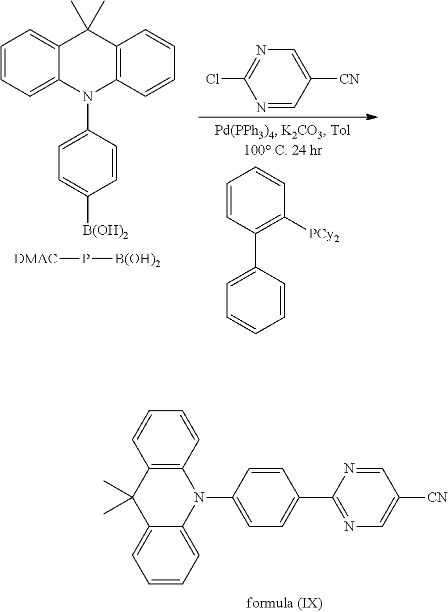

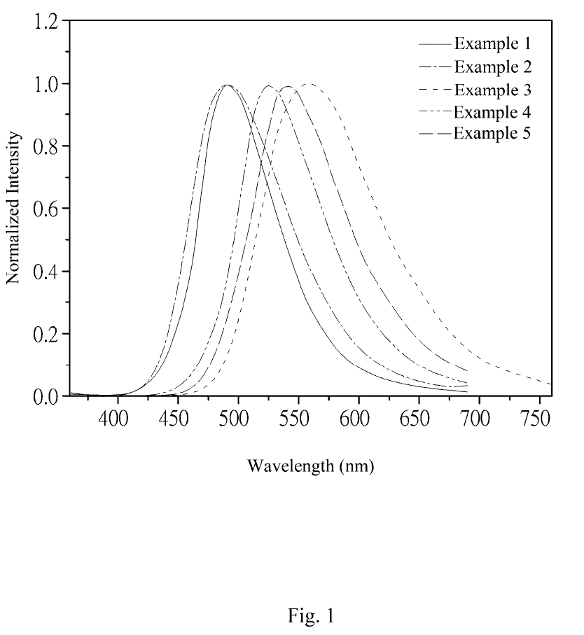

In order to explicitly illustrate the characteristics of the organic fluorescent luminescent material of the present disclosure, the organic fluorescent luminescent materials of Examples 1-5 are detected mainly for photophysical properties and thermal properties. The detection method is described in detail below. Ultraviolet-visible absorption spectra, fluorescence emission spectra, and low temperature phosphorescence spectra of the organic fluorescent luminescent materials of the examples in solution (sol) are measured and their photophysical properties are compared. When absorption spectra and fluorescence spectra in sol are measured, with toluene as solvent and at a sample concentration of 1×10⁻⁴M-1×10⁻⁷M, absorbances in absorption spectra all are within 0.3, and the excitation wavelength in fluorescence spectra is the wavelength of the strongest absorption peak in absorption spectra. Phosphorescence is determined with compounds dissolved in toluene at a low temperature of 77 K. Films of the organic fluorescent luminescent materials of Examples 1-5 are prepared through vacuum evaporation. Samples are doped in mCPCN (having the formula as shown below) at 8 wt % and plated onto a quartz glass with a film thickness of tens of nanometers. Fluorescence spectra of the organic fluorescent luminescent materials of Examples 1-5 in film state are shown in FIG. 1, wherein the vertical axis is normalized intensity (arbitrary unit) and the horizontal axis is wavelength.

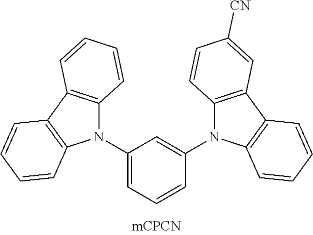 [SMILES: N#Cc1ccc2c(c1)c1ccccc1n2-c1cccc(-n2c3ccccc3c3ccccc32)c1]

In addition, in order to understand thermal stability of the molecules, thermal decomposition temperatures (Td) of the examples are measured by a thermal gravimetric analyzer at a weight loss of 5%. The detection results are shown in Table 1, wherein λ_{abs} is wavelength of absorption peaks, λ_{Fluo} is wavelength of fluorescence emission peaks, λ_{Phos} is wavelength of phosphorescence emission peaks, Eₛ is the lowest energy level of singlet state calculated from fluorescence spectra, E_{T} is the lowest energy level of triplet state calculated from phosphorescence spectra, ΔE_{ST} is Eₛ−E_{T}, i.e. the energy level difference between the lowest energy level of singlet state and the lowest energy level of triplet state, QY is fluorescent quantum yield of the organic fluorescent luminescent materials of Examples 1-5 in film state, and T_{d} is thermal decomposition temperature.

TABLE 1

| | | λ_{Fluo} | λ_{Phos} | | | | | |
|---|---|---|---|---|---|---|---|---|
| | λ_{abs} | (nm) | (nm) | Eₛ | E_{T} | ΔE_{ST} | QY | T_{d} |
| | (nm) | sol/film | sol/film | (eV) | (eV) | (meV) | (%) | (° C.) |
| Exam- | 284, | 488/490 | 502/525 | 2.82 | 2.63 | 190 | 92 | 291 |
| ple 1 | 369 | | | | | | | |
| Exam- | 290, | 504/487 | 490, | 2.84 | 2.66 | 180 | 89 | 273 |
| ple 2 | 384 | | 509/516 | | | | | |
| Exam- | 285, | 562/556 | 508/552 | 2.62 | 2.56 | 60 | 90 | 288 |
| ple 3 | 404 | | | | | | | |
| Exam- | 286, | 519/526 | 515/544 | 2.65 | 2.56 | 90 | 100 | 228 |
| ple 4 | 384 | | | | | | | |
| Exam- | 285, | 543/541 | 502/538 | 2.59 | 2.55 | 40 | 95 | 243 |
| ple 5 | 396 | | | | | | | |

It can be known from Table 1 that, the organic fluorescent luminescent materials of Examples 1-5 all have a relatively high thermal decomposition temperature, 291° C., 273° C., 288° C., 228° C., and 243° C. respectively, which can avoid the decomposition during high temperature evaporation. It can be known from Table 1 and FIG. 1 that, the maximum emission wavelength (λₘₐₓ) of the organic fluorescent luminescent materials of Examples 1-5 ranges from about 487 nm to about 556 nm, so colors of light can be adjusted by changing the substituent X. In addition, Examples 1-5 use DMAC as electron donor, and overall charge transfer capability of the molecules is improved by increasing electron pulling capability of pyridine, such that the organic fluorescent luminescent materials of Examples 1-5 all have a relatively small ΔE_{ST}, 190 meV, 180 meV, 60 meV, 90 meV, and 40 meV, respectively. In other words, the energy level difference between the lowest energy level of singlet state and the lowest energy level of triplet state of the organic fluorescent luminescent materials of Examples 1-5 ranges from about 0 eV to about 0.19 eV. Thus, triplet excitons can be transformed into singlet excitons through thermally activated delayed fluorescence (TADF) mechanism by environmental thermal energy, thereby improving the fluorescence emission efficiency. Therefore, the organic fluorescent luminescent materials of Examples 1-5 all have good fluorescent quantum yield, 92%, 89%, 90%, 100%, and 95%, respectively.

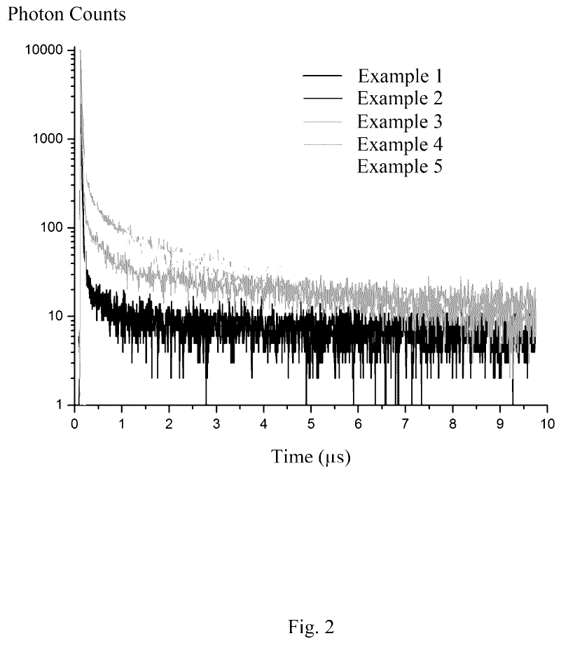

On the other hand, please refer to FIG. 2 and Table 2. FIG. 2 shows transient spectra of the organic fluorescent luminescent materials of Examples 1-5 of the present disclosure in film state at 300 k, wherein the vertical axis is photon counts and the horizontal axis is time. Table 2 is transient fluorescence proportion and delayed fluorescence proportion of the organic fluorescent luminescent materials of the examples, wherein the sum of transient fluorescence proportion and delayed fluorescence proportion is 1, indicating the sum of fluorescence generated from various paths. The transient fluorescence proportion indicates the proportion of fluorescence directly generated by singlet excitons to total fluorescence, and the delayed fluorescence proportion is fluorescence generated by retransforming triplet excitons into singlet excitons with RISC to total fluorescence.

TABLE 2

| | transient fluorescence | delayed fluorescence |
|---|---|---|
| | proportion | proportion |
| Example 1 | 0.93614 | 0.06386 |
| Example 2 | 0.93814 | 0.06186 |
| Example 3 | 0.58192 | 0.41808 |
| Example 4 | 0.70175 | 0.29825 |
| Example 5 | 0.63326 | 0.36674 |

It can be found from Table 2 and FIG. 2 that, Examples 1-2 have some delayed emission signals, and Examples 3-5 have significant delayed emission signals, wherein the delayed emission signals in Examples 3 and 5 are slightly stronger than those in Example 4, which is due to their smaller ΔE_{ST}. As can be seen, for the organic fluorescent luminescent materials of Examples 1-5, triplet excitons can be indeed transformed into singlet excitons through thermally activated delayed fluorescence mechanism by environmental thermal energy (300 K), thereby improving the fluorescence emission efficiency. Because of having a small ΔE_{ST}, the organic fluorescent luminescent materials of Examples 3-5 have more delayed fluorescence.

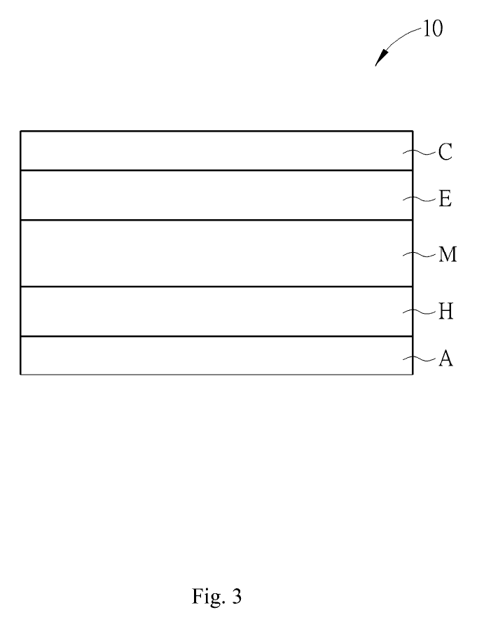

FIG. 3 shows a cross-sectional schematic view of an organic electroluminescent device of the present disclosure. As shown in FIG. 3, the present disclosure further provides an organic electroluminescent device 10, comprising an anode A, a hole transport layer H, an emissive layer M, an electron transport layer E, and a cathode C. The hole transport layer H is disposed on the anode A. The emissive layer M is disposed on the hole transport layer H, wherein the emissive layer M comprises the organic fluorescent luminescent materials of formula (I)-formula (IX), preferably the organic fluorescent luminescent materials of Examples 1-5. In addition, the electron transport layer E is disposed on the emissive layer M. The cathode C is disposed on the electron transport layer E. However, the present disclosure is not limited to the structure above, and the organic electroluminescent device 10 may also optionally comprise a hole injection layer (not shown) and/or an electron injection layer (not shown). In other embodiments, the anode A, the hole transport layer H, the emissive layer M, the electron transport layer E, and the cathode C may be disposed in an inverse order.

For example, actual structures of the organic electroluminescent device 10 are described in detail below.

Experimental Example 1: ITO (60 nm)/PEDOT:PSS (60 nm)/TAPC (10 nm)/mCP (10 nm)/mCPCN: 8 wt % Example 1 (20 nm)/3TPYMB (50 nm)/LiF (1 nm)/Al (100 nm).

Experimental Example 2: ITO (60 nm)/PEDOT:PSS (60 nm)/TAPC (10 nm)/mCP (10 nm)/mCPCN: 8 wt % Example 2 (20 nm)/3TPYMB (50 nm)/LiF (1 nm)/Al (100 nm).

Experimental Example 3: ITO (70 nm)/PEDOT:PSS (70 nm)/TAPC (15 nm)/mCP (10 nm)/mCPCN: 8 wt % Example 3 (20 nm)/3TPYMB (55 nm)/LiF (1 nm)/Al (100 nm).

Experimental Example 4: ITO (70 nm)/PEDOT:PSS (70 nm)/TAPC (10 nm)/mCP (10 nm)/mCPCN: 8% Example 4 (20 nm)/3TPYMPB (55 nm)/LiF (1 nm)/Al (100 nm).

Experimental Example 5: ITO (70 nm)/PEDOT:PSS (70 nm)/TAPC (15 nm)/mCP (10 nm)/mCPCN: 8% Example 5 (20 nm)/3TPYMPB (45 nm)/LiF (0.5 nm)/Al (100 nm).

The emissive layer in Experimental Examples 1-5 comprises 8 wt % Examples 1-5 doped in mCPCN, and the material layers in Experimental Examples 1-5 use commonly used organic materials with the following structures.

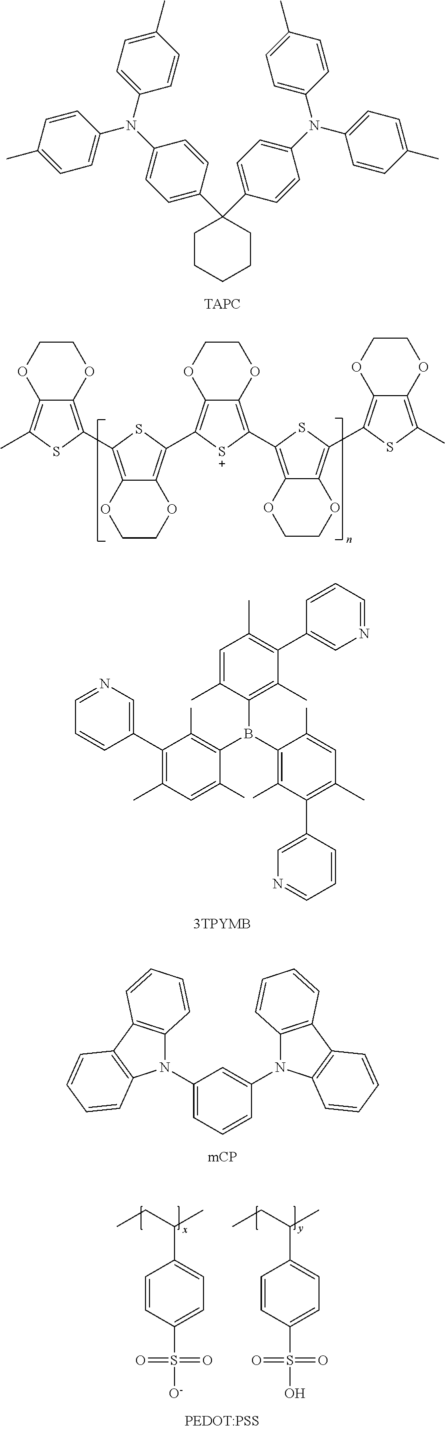

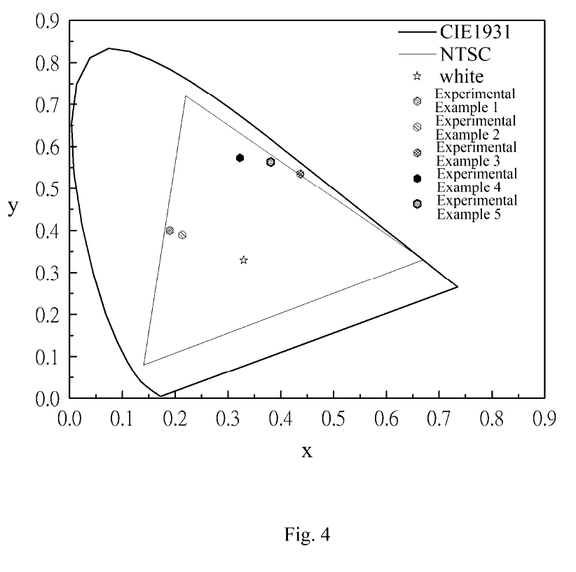

Please refer to FIG. 4 and Table 3. FIG. 4 shows a color coordinate diagram of the organic electroluminescent devices of Experimental Examples 1-5 of the present disclosure, wherein the vertical axis is y and the horizontal axis is x. Table 3 is related data of the organic electroluminescent devices of the experimental examples, wherein Vₒₙ is driving voltage, η_{c,max} is maximum current efficiency, η_{p,max} is maximum power efficiency, η_{ext,max} is maximum external quantum efficiency, L100 (cd/m²) ηₑₓₜ (%) is maximum external quantum efficiency when the organic electroluminescent devices of Experimental Examples 1-5 are at a luminance of L100 cd/m², L1000 (cd/m²) ηₑₓₜ (%) is maximum external quantum efficiency when the organic electroluminescent devices of Experimental Examples 1-5 are at a luminance of L1000 cd/m², and CIE is CIE color coordinate of the organic electroluminescent devices of Experimental Examples 1-5.

TABLE 3

| | | | | | L100 | L1000 | |
|---|---|---|---|---|---|---|---|
| | Vₒₙ | η_{c, max} | η_{p, max} | η_{ext, max} | (cd/m²) | (cd/m²) | |
| | (V) | (cd/A) | (lm/W) | (%) | ηₑₓₜ (%) | ηₑₓₜ (%) | CIE |
| Experi- | 2.1 | 65.6 | 79.3 | 23.9 | 9.89 | 3.49 | (0.23, |
| mental | | | | | | | 0.48) |
| Example 1 | | | | | | | |
| Experi- | 2.1 | 82.4 | 99.5 | 27.3 | 16.9 | 8.16 | (0.25, |
| mental | | | | | | | 0.53) |
| Example 2 | | | | | | | |
| Experi- | 1.6 | 96.3 | 105.5 | 29.2 | 26.5 | 20.6 | (0.43, |
| mental | | | | | | | 0.55) |
| Example 3 | | | | | | | |
| Experi- | 1.9 | 104.5 | 117.2 | 31.1 | 24.0 | 13.1 | (0.30, |
| mental | | | | | | | 0.55) |
| Example 4 | | | | | | | |
| Experi- | 1.9 | 103.7 | 116 | 30.6 | 26.8 | 20.2 | (0.29, |
| mental | | | | | | | 0.59) |
| Example 5 | | | | | | | |

It can be found from Table 3 and FIG. 4 that, when 8 wt % Example 1 is doped, at the driving voltage of 2.1 V, the maximum external quantum efficiency is up to 23.9%, the maximum current efficiency is up to 65.6%, and the CIE color coordinate is (0.23, 0.48). When 8 wt % Example 2 is doped, at the driving voltage of 2.1 V, the maximum external quantum efficiency is up to 27.3%, the maximum current efficiency is up to 82.4%, and the CIE color coordinate is (0.25, 0.53). When 8 wt % Example 3 is doped, at the driving voltage of 1.6V, the maximum external quantum efficiency is up to 29.2%, the maximum current efficiency is up to 96.3%, and the CIE color coordinate is (0.43, 0.55). When 8 wt % Example 4 is doped, at the driving voltage of 1.9V, the maximum external quantum efficiency is up to 31.1%, the maximum current efficiency is up to 104.5%, and the CIE color coordinate is (0.30, 0.55). When 8 wt % Example 5 is doped, at the driving voltage of 1.9V, the maximum external quantum efficiency is up to 30.6%, the maximum current efficiency is up to 103.7%, and the CIE color coordinate is (0.29, 0.59).

According to the aforementioned, the organic electroluminescent devices of Experimental Examples 1-5 have the maximum external quantum efficiency ranging from about 23.9% to about 31.1%. It have excellent external quantum efficiency compared to organic electroluminescent devices with conventional fluorescent materials or conventional phosphorescent materials as emissive layer, and have good fluorescence efficiency and stability because the organic fluorescent luminescent materials of Examples 1-5 are used as the emissive layer M of the organic electroluminescent devices of Experimental Examples 1-5. Thus, the emission efficiency and lifetime of the organic electroluminescent devices of Experimental Examples 1-5 can meet the requirements in practical use.

In sum, the organic fluorescent luminescent material of the present disclosure can effectively reduce ΔE_{ST}, so triplet excitons can be transformed into singlet excitons through thermally activated delayed fluorescence mechanism by environmental thermal energy, thereby improving the fluorescence emission efficiency, and the colors of light can be adjusted by changing the substituent. In addition, the emissive layer of the organic electroluminescent device of the present disclosure use the organic fluorescent luminescent material of the present disclosure, and because of high fluorescence efficiency and good stability of the emissive layer, the emission efficiency and lifetime of the organic electroluminescent device of the present disclosure can meet the requirements in practical use.

The above description only provides preferred embodiments of the present disclosure, and all equivalent changes and modifications made according to the claims of the present disclosure falls within the scope of the present disclosure.

What is claimed is:

1. An organic fluorescent luminescent material represented by the formula (Ar)-(Ph)-(X), wherein Ar is 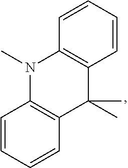 [SMILES: CN1c2ccccc2C(C)(C)c2ccccc21]
Ph comprises at least one benzene ring, X is a six-membered aromatic ring having positions 1, 2, 3, 4, 5 and 6, and at least one nitrogen atom is located on positions 1, 2, 3, 4 or 5, wherein the organic fluorescent luminescent material is represented by the formula as below: 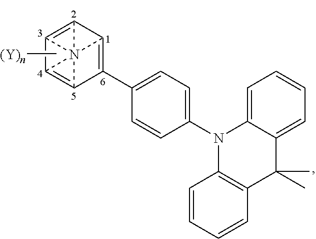
and Y is halogen, a trifluoromethyl group (—CF₃), or a cyanide group (—CN), and n is an integer from 1 to 4.

2. The organic fluorescent luminescent material of claim 1, wherein the organic fluorescent luminescent material is represented by the formula (I):

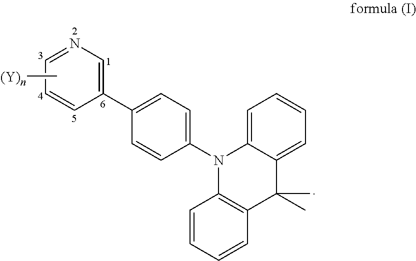

3. The organic fluorescent luminescent material of claim 2, wherein in the formula (I) Y is the cyanide group (—CN) and n is 1, and the organic fluorescent luminescent material is represented by the formula (II):

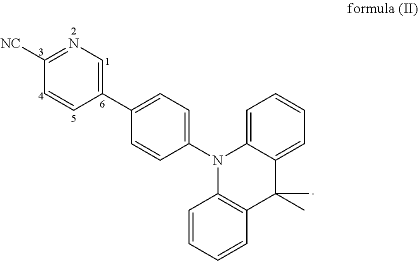 [SMILES: CC1(C)c2ccccc2N(c2ccc(-c3ccc(C#N)nc3)cc2)c2ccccc21]

4. The organic fluorescent luminescent material of claim 1, wherein the organic fluorescent luminescent material is represented by the formula (III): 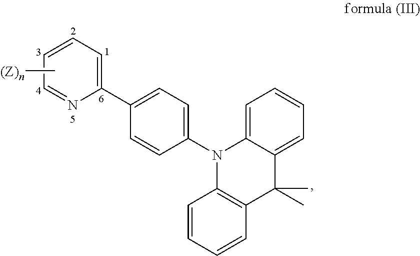
wherein Z is halogen, a trifluoromethyl group (—CF₃), or a cyanide group (—CN), and n is an integer from 1 to 4.

5. The organic fluorescent luminescent material of claim 4, wherein in the formula (III) Z is the cyanide group (—CN) and n is 1, and the organic fluorescent luminescent material is represented by the formula (IV):

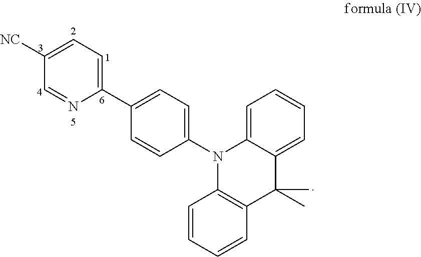 [SMILES: CC1(C)c2ccccc2N(c2ccc(-c3ccc(C#N)cn3)cc2)c2ccccc21]

6. The organic fluorescent luminescent material of claim 4, wherein in the formula (III) Z is the cyanide group (—CN) and n is 2, and the organic fluorescent luminescent material is represented by the formula (V):

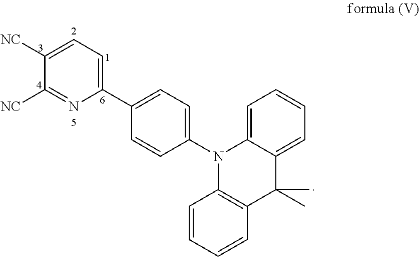 [SMILES: CC1(C)c2ccccc2N(c2ccc(-c3ccc(C#N)c(C#N)n3)cc2)c2ccccc21]

7. The organic fluorescent luminescent material of claim 1, wherein the organic fluorescent luminescent material is represented by the formula (VI): 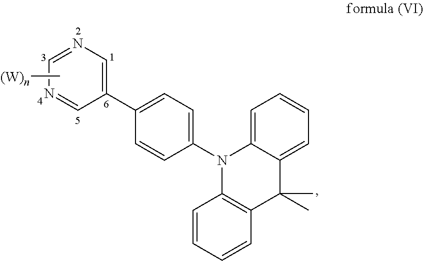
wherein W is halogen, a trifluoromethyl group (—CF₃), or a cyanide group (—CN), and n is an integer from 1 to 3.

8. The organic fluorescent luminescent material of claim 7, wherein in the formula (VI) W is the cyanide group (—CN) and n is 1, and the organic fluorescent luminescent material is represented by the formula (VII):

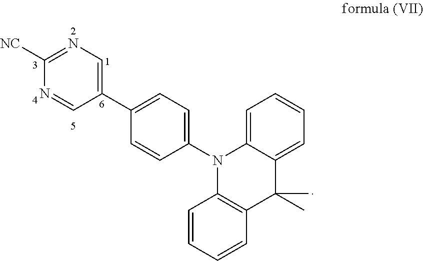 [SMILES: CC1(C)c2ccccc2N(c2ccc(-c3cnc(C#N)nc3)cc2)c2ccccc21]

9. The organic fluorescent luminescent material of claim 1, wherein the organic fluorescent luminescent material is represented by the formula (VIII): 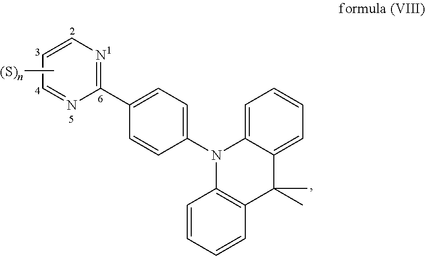
wherein S is halogen, a trifluoromethyl group (—CF₃), or a cyanide group (—CN), and n is an integer from 1 to 3.

10. The organic fluorescent luminescent material of claim 9, wherein in the formula (VIII) S is the cyanide group (—CN) and n is 1, and the organic fluorescent luminescent material is represented by the formula (IX):

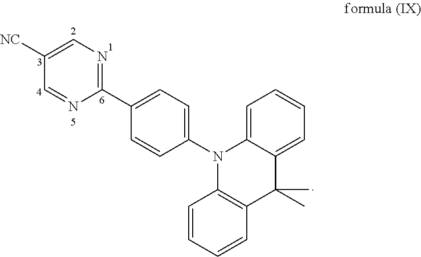 [SMILES: CC1(C)c2ccccc2N(c2ccc(-c3ncc(C#N)cn3)cc2)c2ccccc21]

11. The organic fluorescent luminescent material of claim 1, wherein the energy level difference (ΔE_{ST}) between the lowest energy level of singlet state and the lowest energy level of triplet state of the organic fluorescent luminescent material ranges from about 0 eV to about 0.19 eV.

12. The organic fluorescent luminescent material of claim 1, wherein the maximum emission wavelength (λₘₐₓ) of the organic fluorescent luminescent material ranges from about 487 nm to about 556 nm.

13. An organic electroluminescent device, comprising:
an anode;
a hole transport layer disposed on the anode;
an emissive layer disposed on the hole transport layer, wherein the emissive layer comprises the organic fluorescent luminescent material of claim 1;
an electron transport layer disposed on the emissive layer; and
a cathode disposed on the electron transport layer.

14. The organic electroluminescent device of claim 13, wherein the energy level difference (ΔE_{ST}) between the lowest energy level of singlet state and the lowest energy level of triplet state of the organic fluorescent luminescent material ranges from about 0 eV to about 0.19 eV.

15. The organic electroluminescent device of claim 13, wherein the maximum emission wavelength (λₘₐₓ) of the organic fluorescent luminescent material ranges from about 487 nm to about 556 nm.
